(19) United States
(12) Patent Application Publication
(10) Pub. No.: US 2002/0165596 A1
(43) Pub. Date: Nov. 7, 2002

(54) Bone graft and implantable fusion stimulator positioning device

(76) Inventor: Chris Edward Wilson, Orlando, FL (US)

Correspondence Address: Chris Edward Wilson, MD, 5410 Fairway Road, Fairway, KS, 66205, US

(21) Appl. No.: 09/805,728

(22) Filed: Mar. 14, 2001

(51) Int. Cl. A61N 1/05

(52) U.S. Cl. 607/116

(57) ABSTRACT

A sac composed of woven bio-absorbable suture with an opening (2) for placement of bone graft or bone graft substitutes (5). The device is contoured and sized for positioning in various anatomic areas (FIGS. 3, 4, and 7) and may be coupled to an implantable electronic fusion stimulator device (6 and 7).

### CROSS-REFERENCE TO RELATED APPLICATIONS

Not applicable.

### BACKGROUND

1. Field of Invention

This device relates to bony fusions in the spine and in the long bones. Specifically, the device allows for appropriate dispersal and containment of bone graft and bone graft substitutes as well as for proper positioning of wire leads used in implantable electrical stimulators of bone and fusion healing.

2. Description of Prior Art

The placement of bone graft in spinal fusions and in proximity to fractures is inexact despite careful surgical technique. There is a potential for migration of the graft, unequal distribution of material, and the risk of soft tissue interposition among graft pieces or between the bone and bone graft interface The result of these occurrences is an ineffective surgical procedure and, often, failure of healing of a spinal fusion or of a long bone fracture that has been grafted

Attempts at increasing the rates of success of spinal fusion and grafting of long bone fractures have focused upon the use of autologous bone from the part the supplementation of autologous bone with cadaveric bone, bone graft substitutes, or with chemical bone growth stimulators, and the stimulation of bone formation by electronic devices. Innovation in these areas of study is well represented the orthopaedic literature and in the filing of new patent applications. However, a more precise, reliable, and simple method of placing bone grafting material and electrical fusion stimulator cathodes has been ignored.

The common method of placing graft about the lumbar spine involves exposure of the transverse processes of the individual lumbar levels and the creation of a pocket by dissection of the paralumbar musculature from the deep fascia posterolaterally. In the cervical spine, the individual laminac and spinous processes are cleared of muscle and the pocket for grafting is formed more centrally. For long bones, e.g. a fracture of the tibia, soft tissue is removed in proximity to the fracture site and the freed space is then filled with graft In each of these instances, retractors are placed within the exposure and bone graft is positioned piecemeal. Pitfalls arise first from uneven dispersal of the graft material as the surgeon's view is often obstructed by soft tissue or by bone graft already implanted. Placement of larger quantities of graft often results in migration of material by simple mass effect. Finally, margins of the soft tissue about the pocket are often friable and loose. Soft tissue containment of the graft is frequently inadequate. The result is potential migration of graft over time and soft tissue interposition among the pieces that will prevent solid bony healing.

### SUMMARY

The current invention is a woven bio-absorbable fiber sac that will function as a containment device for autologous bone graft and bone graft substitutes. It may be used in spinal fusions and in the grafting of long bone fractures. It allows a simple, reliable, and more precise method for placement of bone graft materials and for positioning of electrical fusion stimulator implants

### Objects and Advantages

Advantages of such a containment device are:

(a) to provide a single-step method of bone graft placement;

(b) to provide a more reliable distribution of bone graft materials;

(c) to provide a greater area of interface of bone graft with native bone;

(d) to provide a means of concentrating bone graft in targeted areas;

(e) to provide a method of containment of bone graft after the exposure has been closed;

(f) to provide a method for prevention of soft tissue interposition among the graft materials which may prevent healing of bone and produce nonunion.

(g) to provide a method of containment of bone graft fragments which can migrate into proximity with a lumbar or cervical laminectomy and produce injury to the dura or nerve roots;

(h) to provide supplementation and containment of other bone graft materials placed between the containment device and native bone;

(i) to provide a method of placement of cathode leads of an implanted electrical fusion stimulator device,

(j) to provide a method of more accurately positioning metal leads such that electronic fusion stimulators may be used in surgical procedures involving a more limited exposure and a need for precision in lead placement;

(k) to provide a means of allowing close proximity of these leads to one another within the fusion bed, without their contacting one another or nearby hardware, and thereby increasing the effectiveness of the electronic fusion stimulator device.

### DRAWING FIGURES

FIG. 1 shows the absorbable suture lattice of an unfilled containment device with drawstrings untied.

FIG. 2 demonstrates the device filled with bone graft material and drawstrings sealed.

FIG. 3 depicts placement of the device filled with bone graft in the lumbar spine, lateral projection.

FIG. 4 demonstrates proper tandem placement of devices in an in situ lumbar fusion.

FIG. 5 shows the device coupled to an electronic fusion stimulator device with removable power supply.

FIG. 6 demonstrates a similar coupling as FIG. 5, but with power supply meant to remain internal following completion of bone healing.

FIG. 7 depicts a uncontoured device as used in a long bone fracture.

REFERENCE NUMERALS IN DRAWINGS

| | |
|---|---|
| 1 | absorbable suture drawstrings |
| 2 | opening for placement of graft material |
| 3 | bonded outer edge of the sac |
| 4 | drawstring closed |
| 5 | sample single fragment of bone graft material |
| 6 | cathode leads of electrical fusion stimulator |
| 7 | power source of electrical fusion stimulator |
| 8 | long bone fracture interval |

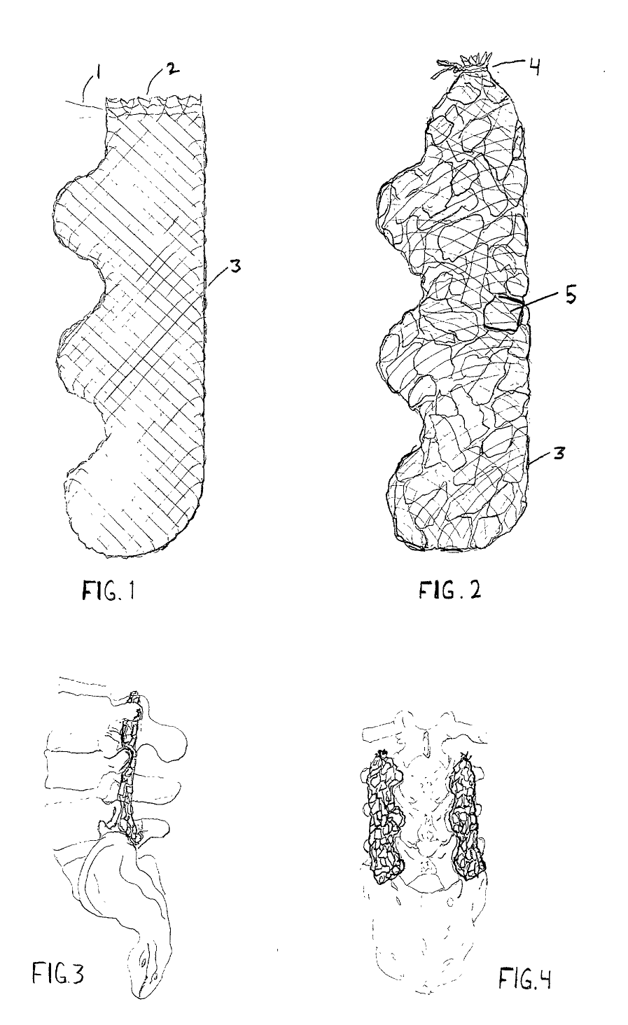

### DESCRIPTION—FIGS. 1-7—PREFERRED EMBODIMENT

A preferred embodiment of the closure of the present invention is illustrated in FIGS. 3, 4, and 7. The bio-absorbable mesh sac has been filled with bone graft material 5 and the device sealed 4 by drawstrings 1 positioned at one end of the device. The invention is then placed in the proximity of desired fusion or fracture healing (FIGS. 3, 4, and 7). In FIG. 7 an implantable source of current 7 with cathode leads 6 have been bonded to the sac in a coordinated arrangement.

FIG. 1 depicts a basic containment device contoured to fit intimately about the posterior lumbar spine. Small caliber absorbable suture, such a Monocryl by Johnson & Johnson, is woven in a “fishnet” manner and bonded about its outer edge 3. The top portion of the sac 2 is left open in the drawing but contains draw-stings 1 that will be closed and tied at the time of surgery 4. Placement of graft material within the sac is aided by a supplied, folded, portion of paper that is removed from the opening at top when the device is filled. The contour of the device has been tailored for placement in the lumbar spine.

FIG. 2 shows the device filled with grafting material 5 (autologous, synthetic, or cadaveric) and the draw-strings closed and tied 4. The device could be delivered sealed by a manufacturer with prepackaged cadaveric or synthetic bone graft material and drawstrings would be omitted. The relative spacing of the absorbable suture weave would be one minimizing content of foreign body but ensuring containment of bony fragments. Again the device shown has been contoured for fit about the lumbar spine and would be used in tandem.

FIG. 3 demonstrates the desired positioning of the device in the lumbar spine in the lateral projection. Note that, if a device prepackaged with graft material is used, the operative surgeon may place free bone beneath the device and still achieve many of the advantages outlined above. Here, the device would function as a blanket barrier holding free bone graft in position. Osteoinductive substances such as Dynagraft (DePuy-Acromed) or Osteofil (Danek) could be intermixed with the bone graft contained in the device or be placed beneath the device in proximity to native bone. Such substances could also be intermixed with the polymer utilized in the manufacture of the suture used in the weave of the device. The sac itself would then allow for containment of bone graft as well as providing chemical stimulation of fusion healing.

FIG. 4 demonstrates the desired positioning of the device in the lumbar spine in the anterior-posterior projection. The sinusoidal central margins of the sac allow continuous contact along the perimeter of the pars interarticularis and facet joints thereby increasing surface area available for bony fusion with reduced risk of fragments piercing the exposed dural sac.

FIG. 5 depicts a prepackaged containment device with allograft or synthetic bone 5 with the addition of an implantable electrical fusion stimulator device 6 & 7. The device illustrated would be appropriately used in tandem for a lumbar fusion spanning four levels. A battery source 7, insulated span or leads, and sinusoidal span of exposed cathodes 6 are shown. Ordinarily, cathode placement is singular on opposite sides of the lumbar exposure and current or electromagnetic fields are hence dissipated. By bonding the flexible cathode leads to the absorbable suture mesh separation of the leads can be reduced without risk of one lead contacting the other. Contact of a metal lead with orthopaedic hardware can also be avoided. Because lead separation can be controlled and current flow focused to within a local area, the effectiveness of the implantable electrical fusion stimulator is markedly improved and a less bulky power supply 7 is possible.

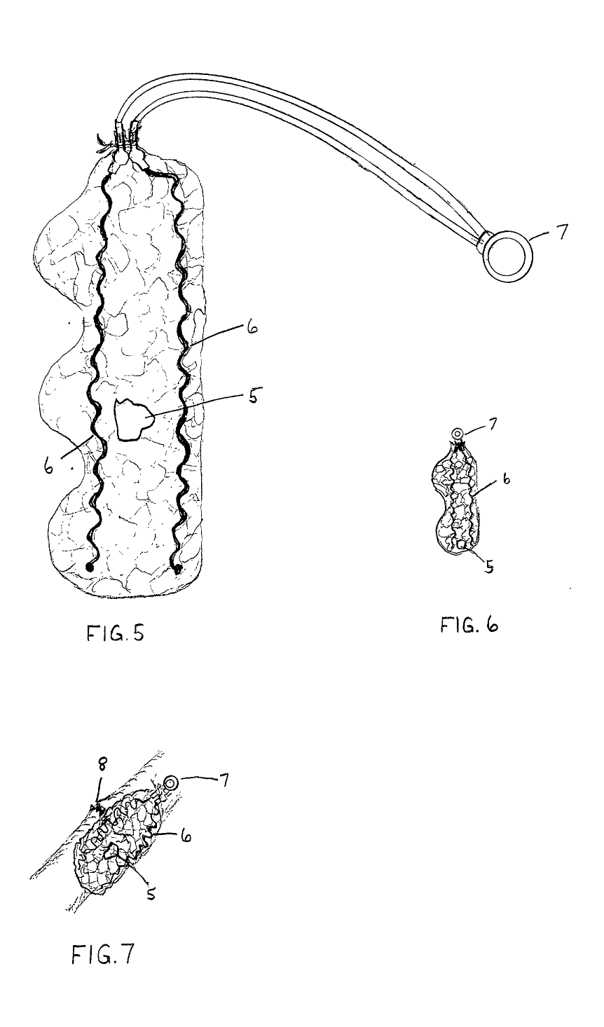

FIG. 6 shows a schematic of figure representing a similar device as seen in figure five but designed to be used in tandem for a lumbar fusion spanning three levels. The implanted battery source 7 would be retained in situ with this construct. The device could be manufactured in various sizes and shapes to allow tailoring of fit for a increased contact of bone graft with native bone.

FIG. 7 demonstrates use of the device for a nonunion of a long bone fracture 8. The fracture interval 8 has been curretted and filled with free graft. The device, with contained bone graft material 5 and electrical fusion stimulator 6 & 7, has been placed over the fracture site and free bone graft. Again, relative size and contour of the device may be adapted to various clinical uses from wrist, cervical, or lumbar fusion to fractures or nonunions of long bones. By allowing precise and focused placement of cathode leads 6, the device permits a smaller battery to be used in the electrical fusion stimulator component. This adaptability and reduction in size will broaden the scope of use of implantable electronic fusion stimulator devices.

### Operation—FIGS. 3,4,7

The manner of use of the bio-absorbable mesh sac involves placement of bone graft material within the open end of the device 2. This may be completed intraoperatively or during the manufacturing process. Once filled with bone graft material 5, drawstrings 1 are closed about the opening 4. The mesh sac may arrive to the operating room coupled with an implantable electronic fusion stimulator consisting of a power source 7 and metal leads 6. These would be bonded to or interwoven with the mesh such that cathode separation and position in relation to the implanted graft are controlled. The device is then inserted in proximity to prepared native bone for the purposes of achieving fusion (FIGS. 3, 4) or fracture healing (FIG. 7):

### Conclusions, Ramifications, and Scope

Accordingly, the reader will see that the containment of bone graft materials by the absorbable mesh device described would allow more reliable and precise placement in the proximity of the desired fusion or area of bony healing. By linkage to an implantable electronic fusion stimulator, the bioabsorbable mesh device also affords more precision in cathode lead placement. The device may be sized and contoured for intimate fit with surrounding bony and soft tissue contours and the mesh fibers may be composed of or impregnated with chemicals stimulating the formation of bone.

I claim:

1. In a sac composed of woven bio-absorbable filaments, bone or bone graft materials are placed through an aperture that is sealed with the sac then containing bone or bone graft materials that are implanted within a desired area of fusion or bony healing.
a. The woven filaments in claim 1 are separated sufficiently to allow contact of sac contents with surrounding tissues but narrowed sufficiently to prevent contents from escaping.
b. The aperature in claim 1 may be closed by drawstrings at the time of surgery or may be sealed by the manufacturer.
c. The sac may arrive filled with autologous bone, cadaveric bone, or artificial bone graft substitutes.
d. The bio-absorbable filaments in claim 1 may be impregnated with or composed of substances stimulating bone formation.
e. The size and contour of the sac in claim 1 may be varied for placement in different anatomic areas of the body.

2. The sac in claim 1 may be coupled to an implantable electronic fusion stimulator device.
a. Cathode leads of the electronic fusion stimulator in claim 2 may be bonded to or intertwined with the bio-absorbable filaments of the sac in claim 1 by the manufacturer or by the surgeon.
